(19) United States
(12) Patent Application Publication
(10) Pub. No.: US 2026/0200661 A1
(43) Pub. Date: Jul. 16, 2026

(54) METHOD FOR MAKING A PACK OF SANITARY ARTICLES

(71) Applicant: TISSUE MACHINERY COMPANY S.P.A. (IN BREVE T.M.C. S.P.A.), Castel Guelfo di Bologna (BO) (IT)

(72) Inventors: Mauro CATTANI, Castel Guelfo di Bologna (BO) (IT); Andrea AVERSA, Castel Guelfo di Bologna (BO) (IT); Michele RASI, Castel Guelfo di Bologna (BO) (IT)

(73) Assignee: TISSUE MACHINERY COMPANY S.P.A. (IN BREVE T.M.C. S.P.A.), Castel Guelfo di Bologna (BO) (IT)

(21) Appl. No.: 19/139,342

(22) PCT Filed: Dec. 15, 2023
(86) PCT No.: PCT/IB2023/062792

§ 371 Date: Jun. 15, 2025

(30) Foreign Application Priority Data
Dec. 16, 2022 (IT) 102022000025842
Dec. 16, 2022 (IT) 102022000025851

(51) Int. Cl. B65D 85/07; A61F 13/15; B65D 75/08; B65D 75/38

(52) U.S. Cl. CPC B65D 85/07; A61F 13/15747; B65D 75/08; B65D 75/38

(57) ABSTRACT

A pack of sanitary articles comprising a casing and a plurality of sanitary articles, each packed in a respective primary packaging. The casing defines a housing cavity obtained by wrapping a sheet material around a wrapping axis. Each of said primary packs comprises an additional sheet material defining at least one closing portion having an elongated shape. The sanitary articles are housed in said cavity in a folded configuration according to at least a first folding line developing along a direction substantially perpendicular to said main direction of development. The first folding line is substantially perpendicular to said wrapping axis of the sheet material forming the casing and substantially perpendicular to said closing portion.

### FIELD OF APPLICATION OF THE INVENTION

The present invention relates to a method for making a pack for packaging several products, in particular sanitary articles of the type intended to be packed folded on themselves.

The invention further relates to a pack containing folded sanitary articles.

### STATE OF THE ART

In the packaging sector, there is a need to provide new packaging solutions which, in particular, allow to limit the use of plastics.

However, meeting such a requirement proves particularly complex in the case of soft sanitary articles, such as absorbent hygiene articles, due to the low structural consistency of the articles.

In fact, in such cases, a possible paper packaging is typically more subject to breakage during product handling with respect to that made of plastic, precisely because of the lower deformability of the packaging material.

Furthermore, the production of packaging is made particularly complex by the deformability of the material, as it is difficult to maintain regular shapes in the pack when wrapping the packaging material around the individual products, or when stuffing them into a possible pre-formed bag.

Such a problem is partly solved by thick cardboard packaging, such as the solutions described in US20130098793A1, i.e., made by means of essentially semi-rigid box-like packaging, but with a significant increase in costs.

Furthermore, such types of solutions are not satisfactorily suited to use in automatic production lines, requiring a number of operating steps which tend to lengthen the packaging times with respect to what are known as ‘flow pack’ solutions typically used for plastic packaging.

In such a context, the Applicant has observed that the packaging solutions of soft sanitary articles, such as those described in US20170057721A1, US20050288644 or U.S. 9,827,150, are not sufficiently well suited to be folded for packaging made of paper.

Consequently, the Applicant has felt the need to identify new solutions for making the packaging of soft sanitary articles which are in particular suitable to be made of both plastic, or paper, without the use of the latter alternative material resulting in a significant drop in the packaging's performance in terms of strength and convenience.

### EXPOSURE AND ADVANTAGES OF THE INVENTION

The technical problem underlying the present invention is therefore that of providing a pack of sanitary articles which allows to improve the known solutions and can at least partially overcome one or more of the identifiable disadvantages in relation to the prior art. In the context of such a problem, an object of the present invention is to provide a pack which lends itself to being made of paper, while maintaining adequate resistance to wear and tear during the normal use of the pack.

A further object of the present invention is to provide a pack and a relative method for making the same which allows a productivity at least comparable to that of the known solutions.

It is also the object of the present invention to provide a pack of sanitary articles which is economical and practical to use.

In its first aspect, such a problem is solved, and one or more of such objects is at least partly achieved by a method for making a pack of sanitary articles which comprises providing a plurality of sanitary articles, preferably soft.

Preferably, it is envisaged to pack each of said sanitary articles in a respective primary packaging, in which said primary packaging defines at least one closing portion having elongated shape.

Preferably, said sanitary articles are packed in said primary packaging with a substantially flat shape, so as to form a respective main development surface of said primary packaging.

The method further envisages preferably providing a sheet material in which an inner surface and an outer surface opposite thereto are defined.

Preferably, it is envisaged to group the sanitary articles next to each other, at the respective main development surface, so as to obtain a stack of sanitary articles.

Preferably, the method envisages approaching said stack of sanitary articles to said sheet material so that said stack of sanitary articles abuts said inner surface of said sheet material.

It is preferably envisaged to wrap said sheet material on said sanitary articles around a wrapping axis, preferably so as to define a joining area extending substantially parallel to said wrapping axis. Preferably at said joining area, two opposite flaps of said sheet material are joined by means of a sealing line.

Preferably, the method envisages folding said sheet material at respective head portions arranged opposite each other along said wrapping axis so as to form respective substantially flat closing surfaces.

Preferably, it is further envisaged to close said head portions so as to obtain a casing within which said sanitary articles are housed.

In fact, the Applicant has observed that when housed in the relative primary packaging, the sanitary articles have more rigid sides with respect to the others. Such sides are typically formed by the sides in which the closing portion is not present.

Consequently, the overall strength of a pack can be improved by envisaging that the casing is wrapped around a stack of sanitary articles and performing the closure by means of a joining area of the two flaps, thus allowing an optimal compaction of the sanitary articles.

In some embodiments, it is in particular envisaged that by means of the relative orientation between the sides of greater strength and the orientation of the sheet material forming the casing, in particular with reference to its wrapping axis around the products. Such an orientation is also particularly advantageous in the step of making the pack, as the sanitary articles are oriented so as to confer high overall structural strength.

The strength of the casing can also be further increased, always thanks to the compaction resulting from the wrapping, also by means of the closure of the head portions obtained by folding the sheet material.

It will be appreciated that in the context of the present invention, the term “wrapping” or “wrapped” referring to a sheet material with respect to one or more products means that the sheet material so as to surround the product or products in a closed-loop configuration, and is in contact at several points with the side surface of the product or set of products surrounded by the material, and preferably with a substantial portion of such a side surface.

Preferably if the product or set of products has a polygonal shape, the material is in contact in at least one point at each of the sides. The wrapping can be obtained either by moving flaps of the sheet material with the products resting thereon, or by moving the products against the sheet material.

Alternatively, the sheet material can be arranged in the closed-loop configuration before the products are positioned in contact therewith, instead inserting them at a later stage.

A further advantage of the invention related to the positioning of the aforesaid fins relates to the creation of the product.

In fact, the pack, and in particular the casing thereof, can be advantageously closed by means of heat-sealing.

Heat-sealing can be used for both the joining area as well as at the head portions. Thanks to the positioning of the fins, which are not in contact with such portions, it is possible to prevent the heat-sealing action from influencing the weld carried out in the closure of the primary packaging, a situation which would instead occur if the closing portions of the latter were in contact with the head portions of the casing during its closure.

It will thus be appreciated that, based on a second aspect, the invention also relates to a pack of sanitary articles, preferably soft, such as absorbent sanitary articles or nappies, comprising a casing and a plurality of said sanitary articles.

Preferably said sanitary articles items are arranged in a stack and preferably each is packed in a respective primary packaging.

Preferably, said sanitary articles are packed in said primary packaging with a substantially flat shape, so as to form a respective main development surface of said primary packaging.

Preferably said casing comprises a sheet material wrapped around said sanitary articles arranged in a stack.

The casing defines a housing cavity preferably obtained by wrapping a sheet material around a wrapping axis.

Preferably, said sheet material is wrapped so as to define a joining area extending substantially parallel to said wrapping axis at which two opposite flaps of said sheet material are joined along a sealing line, so as to create a tubular casing with said sheet material.

Preferably said sheet material comprises an inner surface, facing, in use, towards said cavity, and an outer surface opposite thereto. Preferably at least one of said inner surface and/or said outer surface comprises a heat-sealable layer.

Preferably, the pack comprises primary packaging of said sanitary articles configured so that each sanitary article is individually packed within a corresponding primary packaging.

Preferably said primary packaging comprises an additional sheet material defining at least one closing portion, which preferably has an elongated shape.

Said sanitary articles preferably have an elongated shape so as to define a respective direction of longitudinal development.

Preferably, said sanitary articles are housed in said cavity in a folded configuration according to at least a first folding line.

Preferably said first folding line is substantially perpendicular to said wrapping axis of said sheet material forming the casing.

Preferably said first folding line is substantially perpendicular to said closing portion.

Advantageously, in the pack according to the present invention, the folding of the material is advantageously exploited to give the pack greater structural strength, both in use and when being made.

Preferably said main development surface defines an axis normal to said surface, said normal axis being substantially perpendicular to said wrapping axis.

The aforesaid pack also confers the same advantages as the previous aspect of the invention.

It must be understood that in the context of the present invention the term ‘soft’, referring to sanitary articles, is used to indicate that the article is easily deformable by simple manual action, without exerting any particular force. Such a consistency is preferably achieved by providing an absorbent material inside the article. Typically soft sanitary articles are absorbent sanitary articles or nappies, in which the absorbent material is enclosed between two layers of flexible material, with the ability to suitably anatomically shape themselves during use.

Still in the context of the present invention, the term ‘substantially parallel’ or ‘substantially perpendicular’ referring to a relative orientation between two directions means that such directions are parallel or perpendicular to each other with a possible deviation of ±10°, preferably ±5°.

The term ‘substantially flat’ referring to a sanitary article or other product means that the sanitary article or product has two dimensions which are significantly larger with respect to the third. The product may have both regular and irregular shapes, and the two largest dimensions may define a surface which does not necessarily develop within a plane, but may have irregularities.

The present invention may have at least one of the further preferred features listed below which may advantageously be applied to each of the aforesaid aspects.

The method also preferably comprises folding said sanitary articles along a first folding line.

Thereby, the folding of the sanitary articles can help to ensure that the primary packaging is sufficiently strong to allow the closing portions to be folded during the wrapping of the sheet material onto the sanitary articles.

Preferably said sheet material, when wrapped around said sanitary articles, defines opposite head portions along said wrapping axis, in which at least one of said head portions, and preferably both, is folded back on itself so as to form a substantially flat closing surface of said cavity.

It is thereby possible to contain the overall size of the packaging, as it is possible to avoid the presence of protruding portions where the closure of the cavity occurs, called ‘fins’, by means of a packet closure or similar of the casing.

Preferably, said sheet material comprises an inner surface, facing, in use, towards said cavity, and an outer surface opposite thereto, in which a respective portion of said inner surface is overlapped and joined to a respective portion of said outer surface at an overlapping area.

In some embodiments, said overlapping area is arranged at said substantially flat closing surface.

In some embodiments, said joining area is defined by opposite flaps of said sheet material extending substantially parallel to said wrapping axis.

Thanks to this configuration, it is possible to make a packet formed by means of a sheet material, in which the closing surfaces have a substantially flat shape, benefiting the compactness of the pack as a whole.

In some embodiments, at least one of said respective portions of said inner surface and/or said outer surface comprises a sealable layer. Preferably, at least one of said respective portions of said inner surface and/or said outer surface comprises a heat-sealable layer which, advantageously, forms said sealable layer.

It is thereby possible to achieve the closure of the pack by means of heat-sealing, envisaging a contact between the inner and outer surface of the sheet material and the relative joining. In particular, the presence of a heat-sealable layer on both the inner surface and outer surface is particularly advantageous if the sheet material is paper.

According to preferred embodiments, each head portion defines opposite sides, said opposite sides preferably being folded on each other.

Preferably at least one side of said opposite sides faces said sanitary articles and a further side faces and overlaps in a sandwich-like manner, towards the outside of said pack, said side in an overlapping area, said overlapping area preferably lying on said substantially flat closing surface.

In other words, the closing of the head portions preferably envisages folding said opposite sides being on top of each other, in which a first side of said opposite sides is folded against said sanitary articles at said first folding line and a second side being folded against said first side so as to form a substantially flat closing surface.

It will therefore be appreciated that thanks to the location of the folding line, it is possible to suitably exploit the folding of the sanitary articles to provide a sufficiently rigid abutment surface to be used when closing the pack. It is thereby possible to optimise the process of making the pack.

It should be noted that in the context of the present invention, the term “folded against” will be understood to mean either the presence of a direct contact between the folded side and the sanitary article, in particular its primary packaging, or indirect contact, in which other elements are interposed between the folded side and the sanitary article.

In some embodiments said sanitary article comprises a second folding line substantially parallel to said first folding line.

Preferably said first folding line and/or said second folding line are positioned at end portions of the respective sanitary article when in said folded configuration.

Thereby, the reinforcing effect obtained from folding can be achieved in two different areas of the sanitary article, which advantageously can correspond to opposite peripheral ends.

Preferably, the respective first folding lines of said sanitary articles items develop in a plane substantially perpendicular to said wrapping axis.

Thanks to such an arrangement, all the folding lines form an abutment area for the folded sides, thus providing optimal structural strength.

Preferably said opposite closing portion(s) is/are substantially perpendicular to said first folding lines and/or said second folding lines).

In some embodiments, each of said sanitary articles comprises a single folding line which develops in a direction substantially perpendicular to said wrapping axis when said sanitary articles are in said folded configuration.

In other words, the sanitary article is essentially folded in two, as occurs with nappies, for example. It is understood that there may be other folding lines according to which the product is folded, but with different development with respect to the first folding line. For example, such folding lines may relate to wings of the article or be intended to fold side portions of the article, e.g., if it is panty-shaped.

Thereby, sufficient product strength can be ensured while maintaining a sufficiently flat structure for the sanitary article.

In other embodiments, each of said sanitary articles comprises a total of two folding lines which develop in a direction substantially perpendicular to said wrapping axis when said sanitary articles are in said folded configuration.

In other words, the sanitary article is essentially folded in three, like a wallet, as occurs with absorbent sanitary articles, for example. It is understood that there may be other folding lines according to which the product is folded, but with different development with respect to the first and the second folding line. For example, such folding lines may relate to wings of the article. Also in this case, such a feature allows to ensure sufficient product strength while maintaining a sufficiently flat structure for the sanitary article.

In some embodiments, said casing comprises quick opening means. Preferably the opening means are configured so as to separate said head portion from said casing and define an opening in said casing, the first folding line of said sanitary articles facing towards said opening.

Preferably, said quick opening means include a pre-breaking line. In some embodiments, the pre-breaking line extends peripherally to said casing at a relative side surface of said casing adjacent to said head portion.

The location of the aforesaid quick opening means allows the sanitary articles to be easily available for their removal from the pack, while simultaneously exploiting the structural strength obtained from the folds of the articles to prevent the pack from deforming once opened. In fact, once the pack is opened, the first folding lines of the articles remain exposed, which keep the pack sufficiently stable even once the relative head portion has been removed.

In some embodiments, the pre-breaking line extends at a relative side surface of said casing, different with respect to said substantially flat closing surfaces, preferably in a substantially central area of said side surface.

Thereby, the sanitary articles can be individually removed from the pack through an opening made on a side surface, while still maintaining a sufficiently stable structure for the pack, capable of ensuring a correct housing of the sanitary articles which may still be present therein.

In some embodiments, the quick opening means comprise an openable sealed flap preferably formed at the pre-breaking line.

In preferred embodiments, the openable sealed flap of the casing is sealed by an adhesive, e.g., a resealable adhesive or a non-resealable adhesive and/or sealed by ultrasonic welding or heat sealing.

The term ‘resealable adhesive’ means an adhesive which provides a non-permanent adhesive bond between two adherent surfaces, i.e., a bond which can be broken by applying a tensile force, and recreated by applying a pressure force.

Advantageously, said casing essentially has the a parallelepiped shape, in which said closing surfaces define opposite bases of said parallelepiped and said side surface defines side faces of said parallelepiped, a solution which lends itself particularly well to ensuring a good compactness of the pack, as well as possibly envisaging the aforesaid quick opening means.

In some embodiments, said sanitary articles are housed in said cavity, one rotated 180° about an axis normal to said main development surface with respect to an adjacent sanitary article.

In other embodiments, said sanitary articles are housed in said cavity grouped in at least a first and a second group, the sanitary articles of each group being housed with the same orientation, the sanitary articles of said first group being housed in said cavity rotated 180° about an axis normal to said main development surface with respect to the sanitary articles of said second group. Thanks to such an arranged, in light of the fact that the sanitary articles may not have a symmetrical distribution of the absorbent material present therein, such asymmetry can be exploited to minimise dimensions.

It will also be appreciated that the two distinct distributions of the aforesaid sanitary articles may also be adopted simultaneously in one solution, envisaging one part of the articles arranged grouped and one part of alternating articles.

In some embodiments, said sheet material is made of paper material. Preferably said paper material has a grammage measured according to ISO 536 which is between 25 and 80 grams per square metre, preferably between 35 and 60 grams per square metre, preferably between 45 and 50 grams per square metre.

It will be appreciated that paper materials with a grammage within these ranges can be handled such that they can be easily folded and/or moulded in the pack, while reducing the risk of the paper material being torn or ripped during the production of the pack.

Preferably the sheet material is obtained from a reel, from which the material can be unwound and cut in the required size.

It is thereby possible to make a pack which is particularly advantageous in terms of sustainability and, possibly, keep the possibility of a high degree of automation in the production process. It will be appreciated that the term ‘paper material’, in the context of the present invention, includes any material obtained, for a representative percentage thereof, typically greater than 50% by weight, from cellulose or other fibre of plant origin. In particular, such a term also includes multilayer materials in which at least one material formed by cellulose or another fibre is present.

Furthermore, the term paper material can be understood as a material produced by pressing wet fibres together and drying them into preferably flexible sheets. The fibres can be natural wood fibres, e.g., cellulose-based fibres, bamboo-based fibres and the like. The natural fibres can also be non-wood fibres, such as cotton, abaca, kenaf, sabai grass, flax, esparto grass, straw, jute hemp, bagasse, floss fibres and pineapple leaf fibres. The paper material may consist of a single type of fibre or a mix of different fibres. The paper material may also be treated to better resist moisture, for example with a layer of plastic on the inside or outside. The paper material may consist of a smooth paper, which has been calendared. The paper may be a paper with low porosity. The paper material may include recycled or virgin fibre or a combination thereof.

In some embodiments, said sheet material comprises a plastic material, preferably polyethylene, having a thickness between 20 and 50 μm, preferably between 25 and 45 μm, preferably between 30 and 40 μm.

The flexible polymer sheet may be a polyethylene or polypropylene film or a laminated film. Other non-limiting examples include an LDPE film (low-density polyethylene), LDPE/LLDPE laminate film (linear low-density polyethylene), LDPE/MDPE laminate film (medium-density polyethylene) and LDPE/HDPE laminate film (high-density polyethylene). The film may consist of biodegradable materials, non-biodegradable materials, recyclable materials or virgin material or combinations thereof.

According to some embodiments, the sheet material may have at least one substantially transparent part.

Preferably, said substantially transparent part is located, when the sheet material is wrapped to form the casing, at the closing surfaces, which preferably may coincide with the end walls of the pack.

Thereby, one or both end walls is/are at least substantially transparent so that at least a part of the sanitary articles is visible through the transparent part.

In the context of the present invention, the term ‘transparent’ and its derivatives refer to the ability of the material to transmit light from the inside of a pack to the outside thereof, so that the individually packed absorbent articles for personal hygiene, which are contained in that pack can be seen through that material as clearly as if the material were not present. In this regard, a substantially transparent material is a material in plastic wrap capable of transmitting light in the range from approximately 0% to approximately 25%, as measured by the standard test method ASTM D 1003-11. The substantially transparent part allows the user, for example, to assess the number of absorbent articles for personal hygiene remaining in the pack or to appreciate other aspects (e.g., shapes, colours) of the absorbent articles for personal hygiene.

In some embodiments, for example if the sheet material is polymeric, said inner and/or outer surface of said sheet material at least partially comprises a protective paint. The protective paint can prevent any print from rubbing off the pack.

Preferably, each of said sanitary articles comprises a total of two folding lines which develop in a direction substantially perpendicular to said wrapping axis when said sanitary articles are in said folded configuration.

In preferred embodiments, the sanitary articles have a flat shape, preferably also in the folded configuration. Advantageously, they are housed in said cavity so that a main development surface of a sanitary article faces a corresponding main development surface of an adjacent sanitary article.

Thanks to such a feature, it is possible to obtain a compact structure of the set formed by the plurality of sanitary articles, which optimally lends itself to being wrapped by means of the sheet material in order to be enclosed.

Preferably said wrapping axis is substantially parallel to said closing portion. It will generally be observed that by orienting the closing portions of the primary packaging, which may be in the form of what are called ‘fins’, substantially parallel to the wrapping axis, these will be flattened by means of the sheet material forming the casing, thus containing the overall size of the pack.

Preferably the closing portions are rotated 90° with respect to the folding lines, the former preferably being substantially parallel to the wrapping axis and the latter perpendicular to such an axis.

Further preferred features of the invention will be more generally defined in the dependent claims.

### BRIEF DESCRIPTION OF THE DRAWINGS

These and other features will be more apparent from the following description of certain embodiments illustrated by way of non-limiting example in the accompanying drawings.

FIG. 1 is a perspective view schematically illustrating a pack of sanitary articles according to the present invention;

FIGS. 2 and 3 are two schematic illustrations showing a sanitary article of the pack according to the present invention in a unfolded configuration and a folded configuration, respectively;

FIG. 4 is a perspective view schematically illustrating a sanitary article in the folded configuration enclosed in a relative primary packaging; and

FIG. 5 is a perspective view schematically illustrating the process for making the pack of the present invention;

FIGS. 6A and 6B are two perspective views illustrating quick opening means of the pack according to two different embodiments;

FIGS. 7A and 7B are two perspective views illustrating the extraction of a sanitary article by the quick opening means of FIG. 6B;

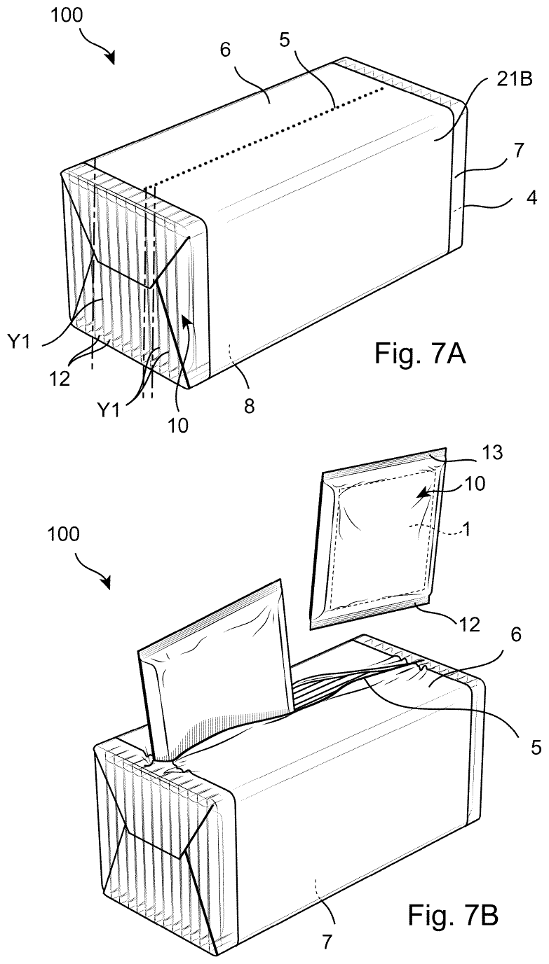

FIG. 8 is a further perspective view schematically illustrating the pack of sanitary articles according to the present invention;

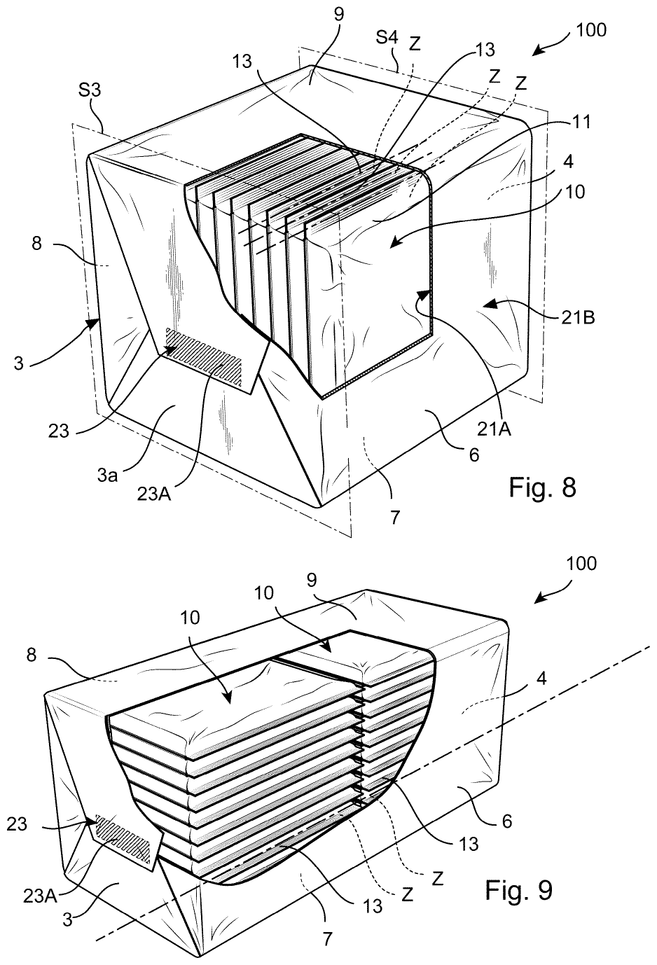

FIG. 9 is a perspective view schematically illustrating the pack of sanitary articles according to the present invention in accordance with an alternative embodiment;

FIGS. 10A to 10G are schematic illustrations illustrating the creation of the pack of sanitary articles according to the present invention;

FIGS. 11A to 11D are schematic illustrations illustrating the creation of the pack of sanitary articles according to the present invention in accordance with an alternative embodiment;

FIG. 12 is a schematic illustration showing the sanitary article and a relative primary packaging in a unfolded configuration.

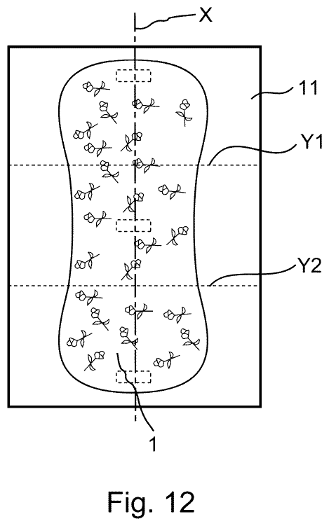

### DETAILED DESCRIPTION OF PREFERRED EMBODIMENTS OF THE INVENTION

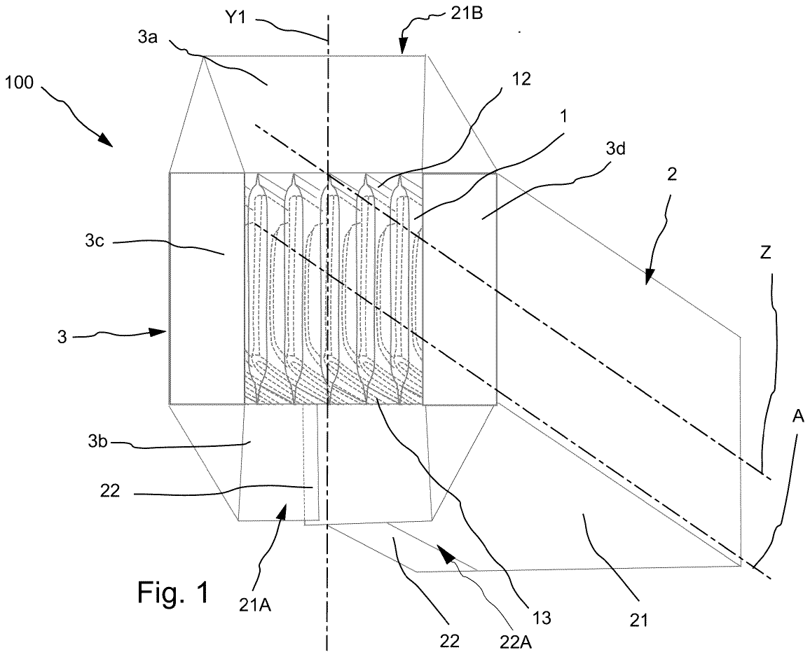

Referring initially to FIG. 1, a pack of sanitary articles 1 is represented overall by the number 100.

As can be seen from the figure, the pack is intended to contain, within a relative casing 2, a plurality of sanitary articles 1.

In the present embodiment example, the sanitary articles 1 are formed by absorbent hygiene articles.

More generally, the sanitary articles can be represented by articles in which an absorbent material is present, not illustrated in the picture, which makes the structure thereof soft.

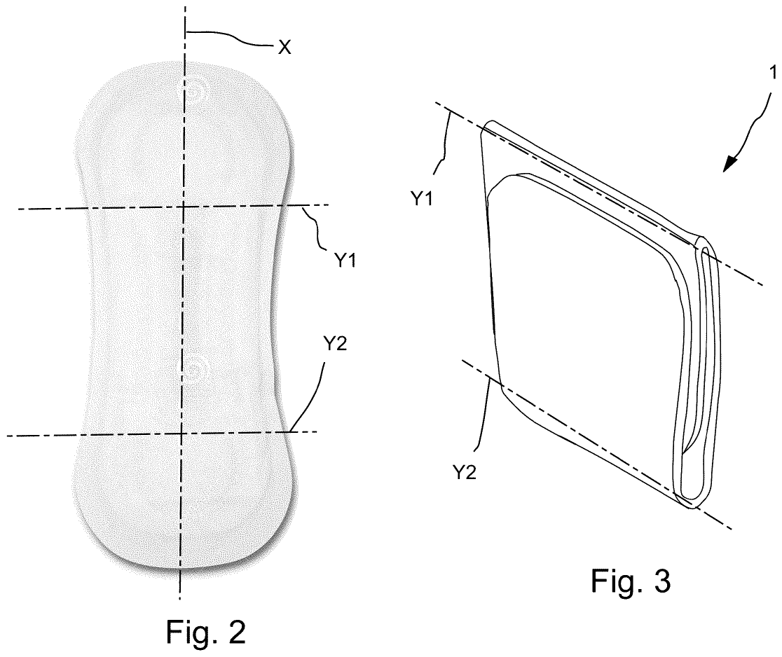

As can be seen from FIGS. 2 and 3, the sanitary articles 1 preferably have an elongated shape so as to define a respective direction of longitudinal development X.

The sanitary articles 1 are used in a substantially unfolded configuration, however, they are advantageously housed in the pack in a folded configuration, illustrated, in a possible example embodiment, in FIG. 3.

The folded configuration can be obtained by folding the sanitary articles along a first folding line Y1 and a second folding line Y2 which preferably develop along a direction substantially perpendicular to the main development direction X.

As can be seen from FIG. 3, the second folding line Y2 is preferably substantially parallel to the first folding line Y1.

It will also be appreciated that, as can be seen in the embodiment example of FIG. 3, the first folding line Y1 and the second folding line Y2 are positioned at the end portions of the respective sanitary article 1 when it is in the folded configuration. In other words, the folding lines define the respective edges of the sanitary article.

It should be noted that although the present description refers to a sanitary article in which two folding lines are present, the present invention may also be applied if only one folding line is present, or if the folding line perpendicular to the main development direction X is associated with further folding lines of different directions.

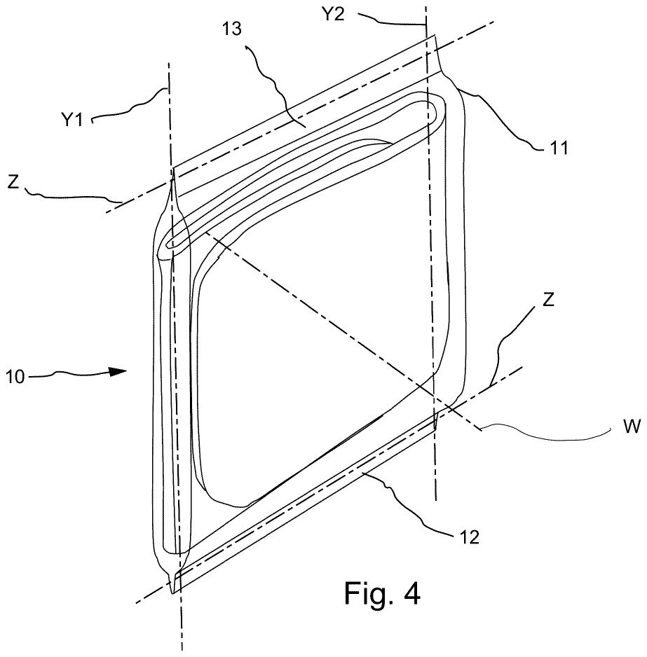

Referring now to FIG. 4, in some embodiments each sanitary article can be housed in a respective primary packaging 10.

The primary packaging 10 can be made by means of a respective sheet material 11 defining one, or preferably two, closing portions 12, 13 which are closed, e.g., by heat-sealing, bagging the article. As can be seen from the figures, if two closing portions are present, these are preferably opposite, i.e., they are arranged on opposite sides of the primary packaging 10.

Preferably, the opposite closing portions 12, 13 have an elongated shape and are substantially perpendicular to the first and to the second folding line Y1, Y2.

Referring now again also to FIGS. 2 and 3, it will be appreciated that in both the unfolded and the folded configuration, the sanitary articles can have a substantially flat shape.

Accordingly, in the primary packaging 10, a main development surface 11, substantially corresponding to the development plane of the sanitary articles when housed in the primary packaging 10, remains defined.

Furthermore, when housed in the pack, and in particular in a relative cavity 20 defined by the same, the sanitary articles 1 may be placed facing each other, at their respective main development surface 11. It is thereby possible to obtain a side-by-side configuration of the sanitary articles, essentially in the form of a stack.

It will be appreciated that a limited number of sanitary articles are represented in the figures, for the sake of illustration clarity.

However, the cavity 2 of the pack 100 of the present invention is intended to be entirely filled with sanitary articles arranged side by side.

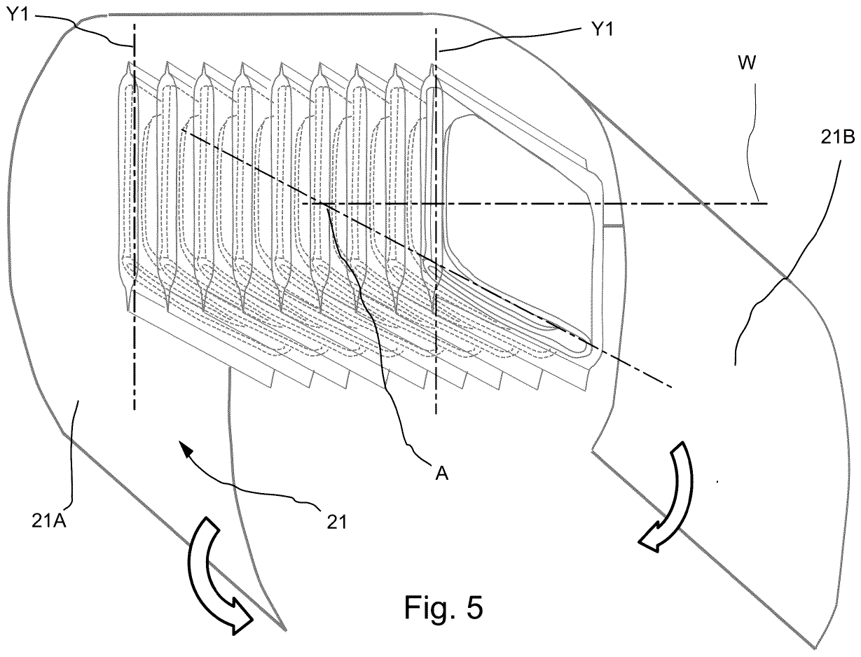

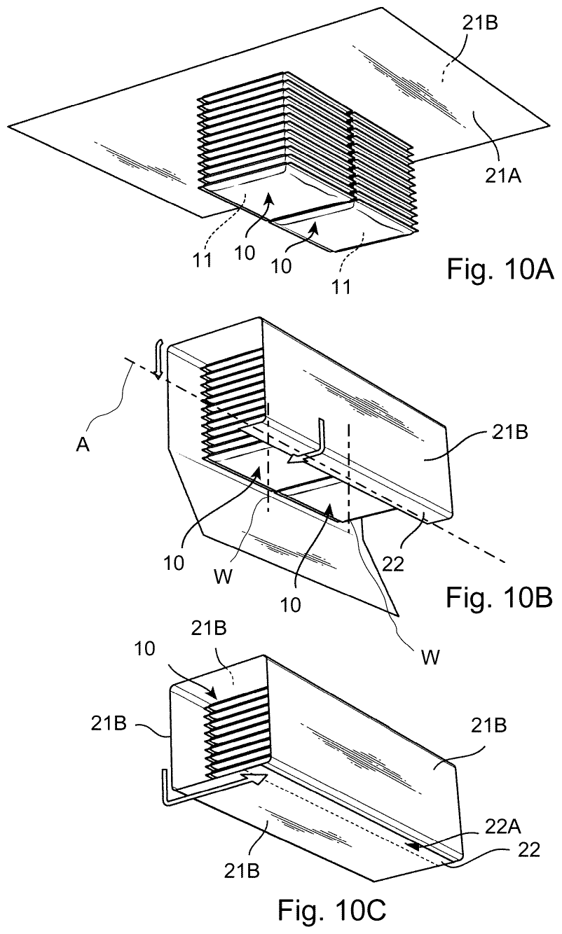

Furthermore, it may be envisaged that several stacks of sanitary articles are present side-by-side in the pack, as for example illustrated in the embodiment of FIG. 9, housed in the cavity 20 according to an arrangement which will be better illustrated below. As will be better illustrated below, and as schematically shown in FIG. 5, the casing 2 can be advantageously obtained by wrapping a sheet material 21 around a wrapping axis A and around the sanitary articles 1.

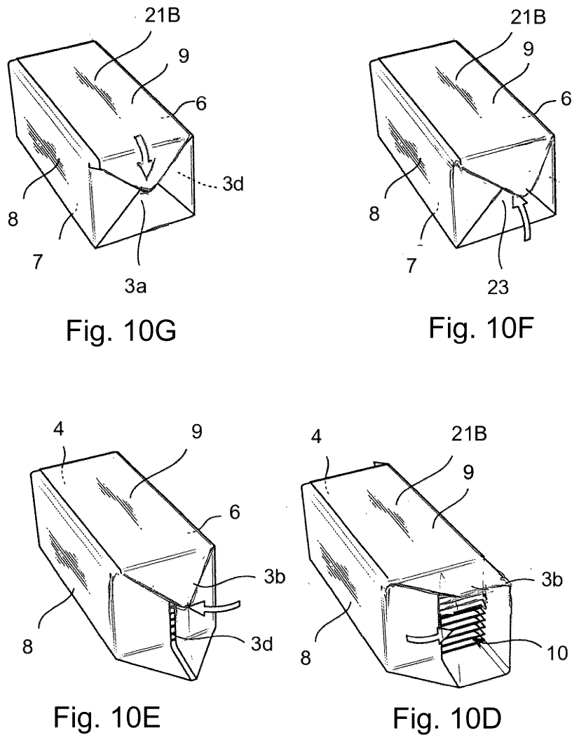

The creation of the pack, with the folding of the sheet material 21, is also illustrated in a first embodiment in FIGS. 10A to 10G.

It will be appreciated, as can be seen in FIG. 10A, that the packs are brought together so that the main development surface of the sanitary articles at the top of the stack abuts a respective flat surface of the sheet material, hereafter referred to as inner surface 21A.

In some embodiments, the stack of sanitary articles 1 is approached to the sheet material 21 by a movement from the bottom upwards.

The sheet material 21 can be made of plastic or, advantageously, paper material.

The sheet material 2 of the pack 1 can be made of materials including, but not limited to, plastic, plastic film, fabric, paper, polymeric films, non-woven and woven layers or fabrics.

In some embodiments, the sheet material is essentially rectangular in shape and comprises transverse and longitudinal edges.

In particular, the sanitary articles are positioned on the sheet material 21 in a folded configuration when this is wrapped to form the cavity 20.

In embodiments in which several stacks of sanitary articles 1 are present, the stacks are preferably side by side along the wrapping axis A, i.e., one following the other along such an axis.

In general, the sanitary articles 1, regardless of the method for making the casing 2, may be housed in said cavity 20 in the folded configuration such that the first folding line Y1, and the second folding line Y2, if present, is substantially perpendicular to the wrapping axis A of the sheet material 21.

In some embodiments, not illustrated in the figures, the sanitary articles 1 may be housed in the cavity 20 each rotated 180° about an axis normal to said main development surface 11 with respect to an adjacent sanitary article 1. In other words, they are placed one inverted with respect to the other.

In other embodiments, again not illustrated in the figures, the sanitary articles 1 may be housed in the cavity 20 grouped into at least a first and a second group. The sanitary articles in each group are housed in the same orientation, while they are reversed between groups. In other words, the sanitary articles 1 of the first group are housed in the cavity 20 are rotated by 180° in relation to the sanitary articles 1 of the second group.

It should be noted that each group may also consist of a single sanitary article 1.

With reference now also to FIG. 10B, following the abutment between the stack of sanitary articles 1 and the sheet material 21, the latter assumes a substantially U-shaped folded configuration. The sheet material is then wrapped entirely around the stack of sanitary articles 1 around the wrapping axis A.

The wrapping occurs by entirely surrounding the sanitary articles 1 by means of the sheet material 21, preferably envisaging a contact between the inner surface 21A of the sheet material and the main development surface 11 of the sanitary article 1 of the opposite end of the stack with respect to that which abuts the sheet material when the stack is approached thereto.

More generally, the contact can be envisaged with one or more points of each side defined by the set formed by grouped sanitary articles.

The wrapping may further envisage an overlapping between two opposite flaps of sheet material 21 at a joining area 22.

In other words, the wrapping of the sheet material 21 on the sanitary articles 1 is preferably performed so that such a joining area 22, in which opposite flaps of sheet material 21 are overlapped, remains defined, which develops substantially parallel to the wrapping axis A. Also in such an area, therefore, the inner surface 21A of the sheet material 21 is in contact with the outer surface 21B thereof.

As will be described in more detail below, the joining between inner surface 21A and outer surface 21B of the sheet material can occur by making a sealing line 22A of one surface on the other, e.g., by heat-sealing.

In preferred embodiments, the sealing line 22A is made by means of a ‘lap seal’ of overlapping flaps, or a ‘fin seal’ of fins.

For this purpose, the inner surface 21A and/or the outer surface 21B of the sheet material 21 may comprise a sealable/heat-sealable layer, for example in the form of a coating.

In preferred embodiments, the transverse edge and/or the transverse edge comprise said sealable/heat-sealable layer. Preferably, the heat-sealable layer may be made in the form of a substantially continuous strip extending along the longitudinal edges and/or longitudinal on the inner surface 21A and/or the outer surface 21B.

In addition to or as an alternative to the essentially continuous strip, the sealable/heat-sealable layer can be applied in a discontinuous coating pattern. Such a pattern may comprise multiple lines, strips, points or other geometric shapes.

Alternatively, the entire inner surface 21A and/or outer surface 21B of the sheet material 21 may be fully coated with the sealable/heat-sealable layer.

It will be appreciated that the use of a heat-sealable layer is particularly advantageous if the sheet material 21 is a paper material. Examples of heat-sealable layers are represented by heat-sealable paint or by heat-sealable glue applied to the inner surface 21A and/or outer surface 21B of the sheet material 21.

In preferred embodiments, the sealable/heat-sealable layer has a grammage between 1 g/m²-5 g/m², preferably 2 g/m²-4 g/m².

As can be seen from the example in FIG. 1, the wrapping of the sheet material 21 not only encloses the sanitary articles 1, but also allows to define opposite head portions 3, 4 along said wrapping axis A.

The head portions 3, 4 are then folded back on themselves to close the cavity 20 with the sanitary articles inside.

The closure can be of the packet type, as will be better illustrated below.

If the pack has a substantially parallelepiped shape, the closure is preferably occurs by making four separate folds of the sheet material 21, with one fold for each of the sides forming one face of the parallelepiped.

It should be noted that if the shape of the pack is parallelepiped, the head portions may form opposite bases of the parallelepiped and will be further defined as side faces 6, 7, 8 and 9.

In general, the fold can be envisaged of at least two opposite sides 3a, 3b, 4a, 4b for each of the head portions 3, 4 of the pack.

A first side 3a, 4a is folded against the sanitary articles 1, preferably against the end portion of the sanitary articles 1 defined by one of the folding lines.

A second side 3b, 4b is instead folded against the first side 3a, 3b, forming a substantially flat closing surface S3, S4.

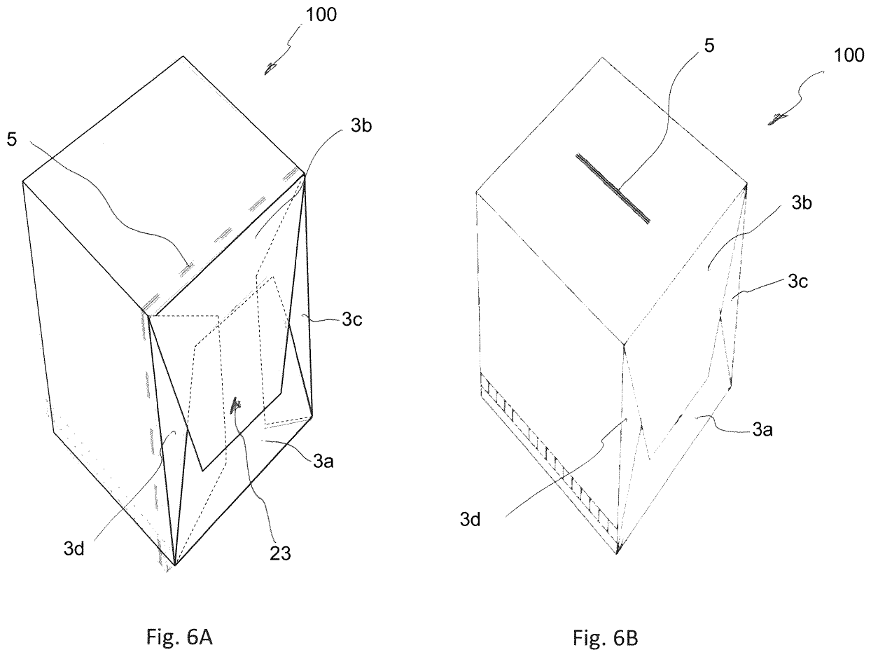

Preferably at least one side 3a, 4a of said opposite sides faces said sanitary articles 1 and a further side 3b, 4b faces and overlaps in a sandwich-like manner, towards the outside of said pack, the side 3a, 4a in an overlapping area 23, the latter illustrated in the example embodiment of FIG. 6A, lying on the substantially flat closing surface of the casing.

It should be noted that in this configuration a respective portion of the inner surface 21A of the sheet material is advantageously overlapped and joined to a respective portion of the outer surface 21B at the overlapping area 23 and, in general, the substantially flat closing surface.

Such joining can occur in a joining area 23A at which the inner surface 21A and outer surface 21B are joined, for example, by heat-sealing or the use of an adhesive.

In the embodiments in which the pack has a substantially parallelepiped shape, it can be envisaged that two further sides 3c, 3d, connecting the two aforesaid sides 3a, 3b, are folded. Such sides can only partially close the pack at the cavity 20, as can be observed in FIG. 1 and FIG. 6A.

Thereby, the casing 2 can substantially have a parallelepiped shape, in which the closing surfaces define opposite bases of the parallelepiped.

As can be seen in FIG. 5, in the case of a packet closure, the wrapping axis A is substantially perpendicular to the first folding line Y1 and possibly the second folding line.

It may therefore be envisaged to fold the opposite sides of the head portions on each other, with at least one side 3a, 4a folded against the sanitary articles 1 at said first folding line Y1 and at least one side 3b, 4b folded against the first side 3a, 3b. Thereby, an substantially flat closing surface can be formed.

Thanks to the presence of the sealable/heat-sealable layer, and in particular to the presence on both the inner surface, and on the outer surface, of the sealable/heat-sealable layer, it is possible to obtain the closure of one side against the other, e.g., by heat-sealing.

However, it is evident that different closure solutions may also be envisaged, for example by heat-sealing the two sides of the head portions, obtaining what is known as the ‘fin’ structure.

However, such a variant is less preferred as it is less advantageous in terms of dimensions.

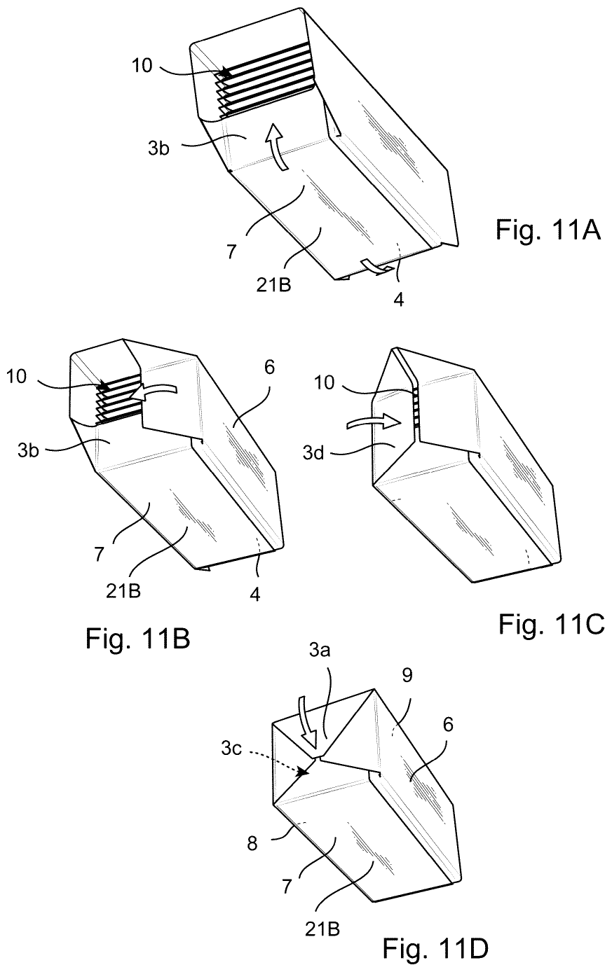

The creation of the pack, with the folding of the sheet material 21, is illustrated in a first embodiment in FIGS. 10A to 10G and in a second embodiment in FIGS. 11A to 11D. Such embodiments differ essentially in the folding sequence of the sides 3a, 3b, 3c and 3d.

In some embodiments, the casing 2 may comprise quick opening means 5 configured so as to define an opening in the casing.

Advantageously, the first folding line Y1 or the second folding line Y2 of sanitary articles 1 faces said opening.

In some embodiments, such as that illustrated in FIG. 6A, the quick opening means 5 can be configured so as to completely or partially separate the head portion 3 from the casing 2 and thus define the aforesaid opening.

By way of example, the quick opening means 5 may include a pre-breaking line preferably extending peripherally to the casing 2 at a relative side surface of said casing 2 adjacent to said head portion 3.

Thus a tab may be present which can be pulled by the user to make the pre-breaking line break.

In some embodiments, such as that illustrated in FIG. 6B, the quick opening means 5 comprise a pre-breaking line extending at a relative side surface of the casing 2, which is different with respect to the substantially flat closing surfaces formed by the folded sides. Preferably such a pre-breaking line can be formed in a substantially central area of such a side surface.

Referring now again to FIG. 5, the pack of the present invention can advantageously be made by first envisaging to provide a plurality of sanitary articles 1 folded along the first folding line Y1. Possibly, if requested, each sanitary article 1 can be housed in a relative primary packaging, as mentioned above.

The creation of the pack then envisages providing the sheet material, after having provided the sanitary articles. The sheet material can advantageously be provided by means of a reel of material which is gradually unwound.

The sheet material is positioned in a stretched configuration, so as to define a relative main plane and a direction normal to said plane, i.e., substantially perpendicular thereto.

The sanitary articles 1 are then placed at the main plane of the sheet material so that the first folding line Y1, and the second folding line, Y2, if present, is substantially parallel to said normal direction of the main plane.

The invention thus solves the proposed problem, while at the same time achieving a plurality of advantages, including the possibility of making a pack entirely by means of paper materials with features and production processes comparable to packs made of plastic.

1. Method for making a pack (100) of sanitary articles (1) comprising:
Providing a plurality of sanitary articles (1), each of said sanitary articles (1) being packed in a respective primary packaging (10), wherein said primary packaging (10) defines at least one closing portion (12, 13) having an elongated shape, said sanitary articles (1) being packed in said primary packaging (10) with a substantially flat shape, so as to form a respective main development surface (11) of said primary packaging (10);
Providing a sheet material (21) in which an inner surface (21A) and an outer surface (21B) opposite thereto are defined;
Grouping the sanitary articles (1) next to each other, at the respective main development surface (11), so as to obtain a stack of sanitary articles (1);
Approaching said stack of sanitary articles (1) to said sheet material (21) so that said stack of sanitary articles (1) abuts said inner surface (21A) of said sheet material (21);
Wrapping said sheet material on said sanitary articles around a wrapping axis (A) so as to define a joining area (22) extending substantially parallel to said wrapping axis (A) at which two opposite flaps of said sheet material (21) are joined along a sealing line (22A), so as to create a tubular casing with said sheet material (21);
Folding the tubular casing formed from said sheet material (21) at respective head portions (3, 4) arranged opposite each other along said wrapping axis (A) so as to form respective substantially flat closing surfaces;
Closing said head portions (3, 4) so as to obtain a closed casing (2) within which said sanitary articles (1) are housed.

2. Method according to claim 1, wherein each head portion (3, 4) defines opposite sides (3a, 3b, 4a, 4b), said closing said head portions (3, 4) comprising joining said opposite sides of the same head portion to each other.

3. Method according to claim 2, wherein a first side (3a, 4a) of said opposite sides faces said sanitary articles (1) at said first folding line (Y1) and a second side (3b, 4b) is folded against said first side (3a, 3b).

4. Method according to any one of the preceding claims, wherein said sanitary articles are folded along a first folding line (Y1).

5. Method according to claim 4, wherein said wrapping axis (A) is substantially perpendicular to said first folding line (Y1).

6. Method according to claim 4 or 5, wherein said sanitary articles are further folded along a second folding line (Y2) parallel to said first folding line (Y1).

7. Method according to any one of claims 4 to 6, wherein the respective first folding lines (Y1) of the sanitary articles (1) of a stack are overall substantially in a plane perpendicular to said wrapping axis (A).

8. Method according to any one of the preceding claims, wherein closing said head portions comprises facing at least one side (3a, 4a) of said opposite sides of each head portion to said sanitary articles (1) and folding a further side (3b, 4b) so as to arrange said further side (3b, 4b) overlapped in a sandwich-like manner on said side (3a, 4a) in an overlapping area (23), said overlapping area (23) lying on said substantially flat closing surface.

9. Method according to any one of the preceding claims, wherein at least one of said respective portions of said inner surface (21A) and/or of said outer surface (21B) comprises a heat-sealable layer, said sealing line (22A) being made by heat-sealing.

10. Method according to any of the preceding claims, comprising providing quick opening means (5) on said sheet material.

11. Method according to claim 10, wherein said quick opening means (5) are configured to allow the separation of one of said head portions (3, 4) from said casing (2) and define an opening in said casing.

12. Method according to claim 11, when claim 10 depends on claim 4, wherein said first folding line (Y1) of said sanitary articles (1) faces said opening.

13. Method according to any one of the preceding claims, wherein said main development surface (11) of said casing defines a respective normal axis (W).

14. Method according to any one of the preceding claims, comprises arranging said sanitary articles (1) so that said normal axis (W) is substantially perpendicular to said wrapping axis (A).

15. Method according to claim 13 or 14, comprising arranging said sanitary articles (1) in said stack each rotated 180° around said normal axis (W) to said main development surface (11) with respect to an adjacent sanitary article (1).

16. Method according to the preceding claim, comprising arranging said sanitary articles (1) in said stack grouped in at least a first and a second group, the sanitary articles of each group being housed with the same orientation, the sanitary articles (1) of said first group being rotated 180° about an axis normal to said main development surface (11) with respect to the sanitary articles (1) of said second group.

17. Method according to any one of the preceding claims, wherein said sheet material (21) is made of paper material, with a weight between 25 and 80 grams per square metre, preferably between 35 and 60 grams per square metre, more preferably between 45 and 50 grams per square metre.

18. Method according to any one of claims 1 to 16, wherein said sheet material (21) comprises a plastic material, preferably polyethylene, having a thickness between 20 and 50 μm, preferably between 25 and 45 μm, more preferably between 30 and 40 μm.

19. Method according to any one of the preceding claims, wherein said sanitary articles (1) comprise an absorbent material and are preferably soft.

20. Method according to any one of the preceding claims, wherein each of said sanitary articles (1) comprises a total of two folding lines developing in a direction substantially perpendicular to said wrapping axis (A).

21. Method according to any one of the preceding claims, wherein said sheet material (21) is folded into a U-shape following the abutment with said stack of sanitary articles (1).

22. Method according to any one of the preceding claims, wherein said stack of sanitary articles (1) are brought closer to said sheet material (21) by means of movement of said stack of sanitary articles (1) from the bottom upwards along a direction substantially perpendicular to a plane on which said sheet material (21) lies.

23. Method according to any one of the preceding claims, wherein said stack of sanitary articles (1) abuts said sheet material (21) at a main development surface of a respective sanitary article (1) arranged at an end of the stack.

24. Method according to the preceding claim, wherein said joining area (22) is arranged at a respective main development surface (11) of a respective sanitary article (1) arranged at an opposite end of the stack with respect to the end abutting said sheet material (21).

25. Method according to any one of claims 1 to 21, wherein said stack of sanitary articles (1) abuts said sheet material (21) at respective edges of said sanitary articles (1) arranged in a stack, said edges not coinciding with the respective closing portions (12, 13).

26. Method according to any one of the preceding claims, wherein said opposite flaps of said sheet material (21) are overlapped at said joining area (22).

27. Method according to any one of the preceding claims, wherein said wrapping axis is substantially parallel to said closing portion (12, 13).

28. Pack (100) of sanitary articles (1) comprising a casing (2) and a plurality of sanitary articles (1) arranged in a stack, each packed in a respective primary packaging (10), wherein:
said sanitary articles (1) are packed in said primary packaging (10) with a substantially flat shape so as to form a respective main development surface (11) of said primary packaging (10);
each of said primary packaging (10) comprises an additional sheet material (11) defining at least one closing portion (12, 13) having an elongated shape;
said casing (2) comprises a sheet material (21) wrapped around said sanitary articles (1) arranged in a stack, said sheet material (21) being wrapped so as to define a joining area (22) extending substantially parallel to said wrapping axis (A) at which two opposite edges of said sheet material (21) are joined along a sealing line (22A), so as to create a tubular casing with said sheet material (21), a housing cavity (20) being defined in said casing in which said sanitary articles (1) are arranged;
said sheet material (21) comprises an inner surface, facing, in use, towards said cavity, and an outer surface opposite thereto, at least one of said inner surface and/or said outer surface comprises a heat-sealable layer;
said sheet material (21), when wrapped around said sanitary articles (1), defines opposite head portions (3, 4) along said wrapping axis, wherein at least one of said head portions, and preferably both, is folded on itself in such a manner as to form a substantially flat closing surface (S3, S4) of said housing cavity (20);
said main development surface (11) defines a normal axis (W) to said surface, said normal axis (W) being substantially perpendicular to said wrapping axis (A).

29. Pack (100) of sanitary articles (1) according to the preceding claim, wherein said sanitary articles (1) have an elongated shape so as to define a respective direction of longitudinal development (X), and being housed in said cavity (20) in a folded configuration according to at least a first folding line (Y1) developing along a direction substantially perpendicular to said main direction of development (X), said first folding line (Y1) being substantially perpendicular to said wrapping axis (A) of said sheet material (21) forming the casing (2) and substantially perpendicular to said closing portion (12, 13);

30. Pack (100) of sanitary articles (1) according to claim 28, wherein said stack of sanitary articles (1) are arranged so that said joining area (22) abuts a main development surface of a respective sanitary article (1) arranged at one end of the stack.
